(19) United States
(12) Patent Application Publication
(10) Pub. No.: US 2026/0069177 A1
(43) Pub. Date: Mar. 12, 2026

(54) METHOD AND SYSTEM FOR ANALYZING AND TREATING MENTAL CONDITIONS OF A HUMAN SUBJECT BASED ON EYE TRACKING

(71) Applicant: MIND HELP LIMITED, Wallsend (GB)

(72) Inventor: Edward Matthew HUDSON, Durham (GB)

(73) Assignee: MIND HELP LIMITED, Wallsend (GB)

(21) Appl. No.: 19/393,606

(22) Filed: Nov. 19, 2025

Related U.S. Application Data
(63) Continuation of application No. 17/155,737, filed on Jan. 22, 2021, now abandoned.
(60) Provisional application No. 62/964,175, filed on Jan. 22, 2020.

(51) Int. Cl. A61B 5/16; A61B 5/00

(52) U.S. Cl. CPC A61B 5/163; A61B 5/165; A61B 5/7405; A61B 5/742; A61B 5/7475

(57) ABSTRACT

A method and a system for analyzing and treating mental conditions of a human subject based on eye tracking are provided herein. The method may include: receiving human subject data associated with a mental or a physical condition related to a human subject; and instructing the human subject, via a user interface, to look at a specific location, while thinking about something associated with the mental or physical condition, wherein the specific location is automatically selected by a computer processor, based on said human subject data.

### CROSS REFERENCE TO RELATED APPLICATIONS

This application is a Continuation of U.S. patent application Ser. No. 17/155,737, filed on Jan. 22, 2021. which claims the benefit of U.S. Provisional Patent Application No. 62/964,175, filed on Jan. 22, 2020, which is incorporated herein by reference in its entirety.

### FIELD OF THE INVENTION

The present invention relates generally to the field of data processing and, more particularly, to processing of human mental and physical condition using eye tracking.

### BACKGROUND OF THE INVENTION

Stress and worry are driven by how a person perceives his/her problems, and it is always subjective whether the information is available to the person via their senses (bottom up processing) or based upon what they know and prior experience (top down processing). We are able to see that the brain redistributes masses of energy during stressful situations, but why this happens is still not yet fully understood.

Some mental and physiological conditions affect the way humans subconsciously behave. Specifically, mental and physiological conditions may have an impact on the way humans respond to a given stimulation. However, associating the response of a specific human subject with the respective mental condition is far from trivial.

One of the challenges is finding a correlation between the non-verbal change of a human subject and a given stimulation presented to him or her.

Another challenge is being able to detect non-verbal changes and analyzing them in a way that provides them with a meaningful manner that can be used for therapeutic or educational purposes.

### SUMMARY OF THE INVENTION

Some embodiments of the present invention address the aforementioned challenges by providing a computerized platform capable of generating a challenge to a human subject that is directly associated with one or more mental or physical conditions affecting the human subject. The computerized platform then detects the non-verbal response to the challenge in a manner that provides insight to the nature of the mental condition.

Some embodiments of the present invention provide a method and a system for analyzing and treating mental and/or physical conditions of a human subject based on eye tracking. The method may include: receiving human subject data associated with a mental or a physical condition related to a human subject; and instructing the human subject, via a user interface, to look at a specific location, while thinking about something associated with said mental or physical condition, wherein the specific location is automatically selected by a computer processor, based on said human subject data.

Advantageously, some embodiments of the present invention provide a non-intrusive, subconsciousness interrogation process that enables deducing the mental or physiological condition of a person, including classification thereof, without verbally interacting with the human subject. By doing so, the level of cooperation and collaboration required from the human subject is significantly reduced. This leads to a much more efficient diagnosis process.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram illustrating a non-limiting exemplary architecture of a system in accordance with some embodiments of the present invention; and

FIG. 2 is a high-level flowchart illustrating a non-limiting exemplary computer-implemented method in accordance with some embodiments of the present invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Unless specifically stated otherwise, as apparent from the following discussions, it is appreciated that, throughout the specification, discussions utilizing terms such as “processing”, ‘computing”, “calculating”, “determining”, or the like refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

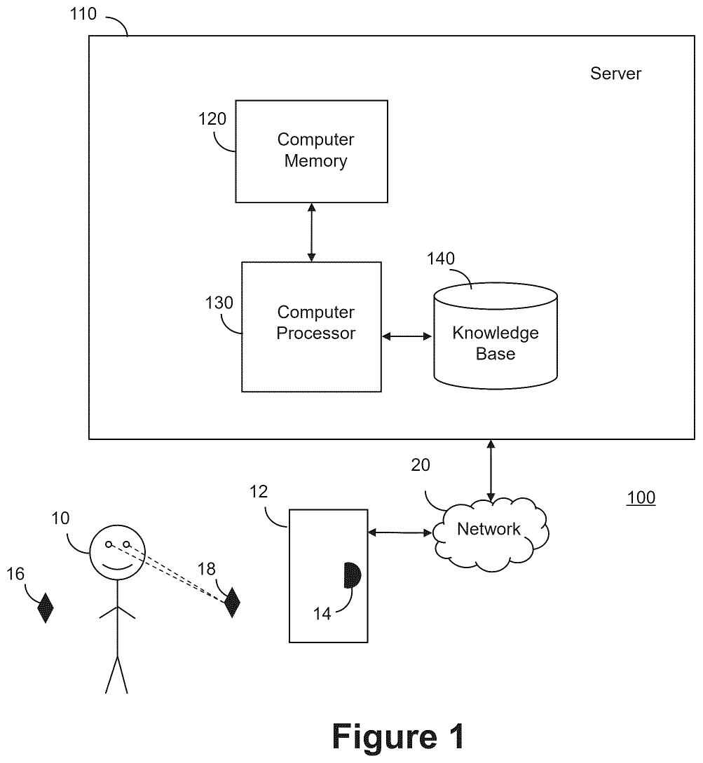

FIG. 1 is a block diagram illustrating non-limiting exemplary architecture of a system 100 in accordance with some embodiments of the present invention. System 100 may include a computer memory 120 configured to receive human subject data comprising a mental condition related to a human subject. The mental condition may include stress, anxiety, fear, post-traumatic stress disorder and the like. System 100 may further include a computer processor configured to automatically select a specific location based on said human subject data. System 100 may further include a user interface 12 configured to instruct the human subject, to look at a specific location 18, while thinking about the mental condition.

Computer processor 130 and computer memory 120 may be located on a server 110 which may be connected to user interface 12 via a network 20.

In accordance with some embodiments of the present invention, the receiving of the human subject data may be carried out by instructing the human subject to discuss an issue or answer a question regarding a physical condition or the mental condition thereof, while monitoring, using a sensor such as an eye tracker 14, a change in line of sight of the human subject towards a representative location 16.

In accordance with some embodiments of the present invention, a specific location 18 may be calculated further based on representative location 16.

In accordance with some embodiments of the present invention, system 100 may further include a knowledge base 140, wherein specific location 18 may be calculated further based on knowledge base 140 which associates representative locations with respective mental conditions.

In accordance with some embodiments of the present invention, the receiving of said human subject data may be carried out by: presenting a challenge to said human subject; capturing, via sensor 14, an external non-verbal physical change of said human subject, responsive to said challenge; and analyzing, via computer processor 130, said change, in order to detect the mental condition affecting said human subject.

In accordance with some embodiments of the present invention, user interface 12 may include a display, wherein the specific location may be a point on the display or a direction.

According to some embodiments of the present invention, the challenge may include a question contextually associated with the psychological condition.

According to some embodiments of the present invention, the physical change may include facial movements or gestures of human subject 10.

According to some embodiments of the present invention, the physical change may include a change in a direction of a gaze of human subject 10.

According to some embodiments of the present invention, sensor 14 may be a camera, wherein the physical change is recorded as an image or a sequence of images.

According to some embodiments of the present invention, user interface 12 may be an electronic display.

According to some embodiments of the present invention, user interface 12 and sensor 14 are components of a single device such as a smartphone.

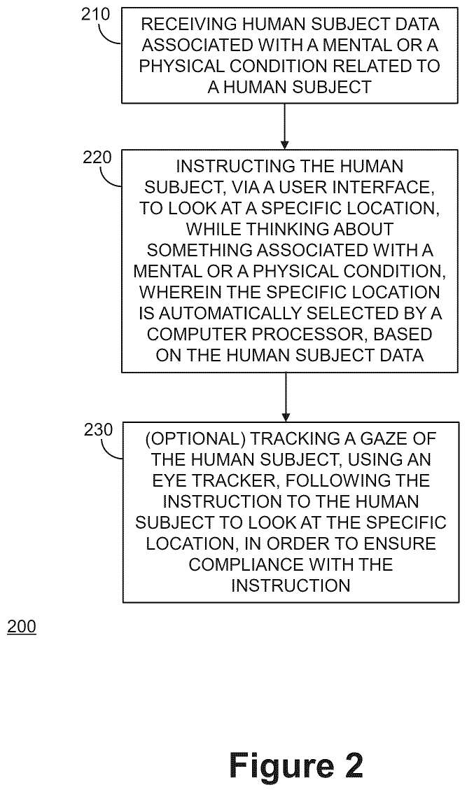

FIG. 2 is a high-level flowchart illustrating a non-limiting exemplary method 200 in accordance with some embodiments of the present invention. Method 200 may include the following two steps: receiving human subject data comprising a mental condition related to a human subject 210; and instructing the human subject, via a user interface, to look at a specific location, while thinking about something associated with the mental or the physical condition, wherein the specific location is automatically selected by a computer processor, based on said human subject data 220.

In accordance with some embodiments of the present invention, the receiving of the human subject data is carried out by instructing the human subject to discuss issues or answer questions regarding a physical condition or the mental condition thereof, while monitoring a change in line of sight of the human subject towards a representative location.

In accordance with some embodiments of the present invention, the specific location may be calculated further based on the representative location.

In accordance with some embodiments of the present invention, the specific location may be calculated further based on a knowledge base which associates representative locations with respective mental conditions.

In accordance with some embodiments of the present invention, the receiving of the human subject data may be carried out by: presenting a challenge to said human subject; capturing, via a sensor, an external non-verbal physical change of said human subject, responsive to said challenge; and analyzing, via said computer processor, said change, in order to detect the mental condition affecting said human subject.

In accordance with some embodiments of the present invention, the automatic selection of the specific location may be carried out based on a knowledge base associating a plurality of types of mental conditions and a plurality of specific locations.

In accordance with some embodiments of the present invention, method 200 may further include the step of tracking a gaze of the human subject, using an eye tracker, following the instruction to the human subject to look at the specific location, in order to ensure compliance with the instruction 230.

In accordance with some embodiments of the present invention, method 200 may further include providing the human subject with a mental challenge prior to instructing the human subject to look at the specific location, wherein the mental challenge relates to the mental condition thereof.

In accordance with some embodiments of the present invention, method 200 may further include providing the human subject with a mental challenge prior to instructing the human subject to look at the specific location, wherein the mental challenge is selected so as to enhance curiosity in said human subject.

In accordance with some embodiments of the present invention, method 200 may further include providing the human subject with a visual and/or audio feedback upon detecting a gaze thereof at the specific location.

In accordance with some embodiments of the present invention, method 200 may further include providing the human subject with a visual and/or audio feedback upon detecting that the human subject Is looking away from the specific point where the human subject should be looking. This is important to making the human subject aware of his/her eye movements in order to ensure that he/she does not look away.

In accordance with some embodiments of the present invention, the visual and/or audio feedback/stimulation is selected so as to enhance a positive mental reaction at the human subject. The feedback/stimulation can be carried out upon the instruction to the human subject to look at the specific point or only upon detecting the gaze of the human subject to be aligned with the specific point.

In accordance with some embodiments of the present invention, the specific location may be a point in an environment in which the human subject is present or alternatively a direction such as “bottom right”.

In accordance with some embodiments of the present invention, the user interface may include a display, and the specific location may be a point on the display.

It should be noted that method 200, according to some embodiments of the present invention, may be stored as instructions in a computer readable medium that cause processors, such as central processing units (CPU), to perform the method. Additionally, the method described in the present disclosure can be stored as instructions in a non-transitory computer readable medium, such as storage devices which may include hard disk drives, solid state drives, flash memories, and the like. Additionally, non-transitory computer readable medium can be memory units.

In order to implement the method according to some embodiments of the present invention, a computer processor may receive instructions and data from a read-only memory or a random-access memory or both. At least one of aforementioned steps is performed by at least one processor associated with a computer. The essential elements of a computer are a processor for executing instructions and one or more memories for storing instructions and data. Generally, a computer will also include, or be operatively coupled to communicate with, one or more mass storage devices for storing data files. Storage modules suitable for tangibly embodying computer program instructions and data include all forms of non-volatile memory, including by way of example semiconductor memory devices, such as EPROM, EEPROM, and flash memory devices and also magneto-optic storage devices.

As will be appreciated by one skilled in the art, some aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system. ” Furthermore, some aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, JavaScript Object Notation (JSON), C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some aspects of the present invention are described above with reference to flowchart illustrations and/or portion diagrams of methods, apparatus (systems) and computer program products according to some embodiments of the invention. It will be understood that each portion of the flowchart illustrations and/or portion diagrams, and combinations of portions in the flowchart illustrations and/or portion diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram portion or portions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram portion or portions.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram portion or portions.

The aforementioned flowchart and diagrams illustrate the architecture, functionality, and operation of possible implementations of systems, methods and computer program products according to various embodiments of the present invention. In this regard, each portion in the flowchart or portion diagrams may represent a module, segment, or portion of code, which comprises one or more executable instructions for implementing the specified logical function(s). It should also be noted that, in some alternative implementations, the functions noted in the portion may occur out of the order noted in the figures. For example, two portions shown in succession may, in fact, be executed substantially concurrently, or the portions may sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the inventions. The various appearances of “one embodiment,” “an embodiment” or “some embodiments”do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional elements.

It is to be understood that, where the claims or specification refer to “a” or “an” element, such reference is not be construed that there is only one of that elements.

It is to be understood that, where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention may be implemented in the testing or practice with methods and materials equivalent or like those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method comprising:
receiving human subject data associated with a mental or a physical condition related to a human subject; and
instructing the human subject, via a user interface, to look at a specific location, while thinking about something associated with said mental or physical condition,
wherein the specific location is automatically selected by a computer processor, based on said human subject data.

2. The method according to claim 1, wherein receiving of said human subject data is carried out by instructing the human subject to answer a question regarding the physical or the mental condition thereof, while monitoring a change in line of sight of the human subject towards a representative location.

3. The method according to claim 2, wherein said monitoring a change in line of sight of the human subject towards a representative location is carried out using an eye tracker.

4. The method according to claim 2, wherein the specific location is calculated based on the representative location.

5. The method according to claim 4, wherein the specific location is calculated further based on a knowledge base which associates representative locations with respective mental conditions.

6. The method according to claim 1, wherein the receiving of said human subject data is carried out by: presenting a challenge to said human subject; capturing, via a sensor, an external non-verbal physical change of said human subject, responsive to said challenge; and analyzing, via said computer processor, said change, in order to detect the mental condition affecting said human subject.

7. The method according to claim 1, wherein the automatic selection of the specific location is carried out based on a knowledge base associating a plurality of types of mental conditions and a plurality of specific locations.

8. The method according to claim 1, further comprising: tracking a gaze of said human subject, using an eye tracker, following the instruction to the human subject to look at the specific location, in order to ensure compliance with said instruction.

9. The method according to claim 1, further comprising: providing the human subject with a mental challenge prior to instructing the human subject to look at the specific location, wherein the mental challenge relates to the mental condition thereof.

10. The method according to claim 1, further comprising: providing the human subject with a mental challenge prior to instructing the human subject to look at the specific location, wherein the mental challenge is selected so as to enhance curiosity in said human subject.

11. The method according to claim 1, further comprising: providing the human subject with a visual and/or audio feedback upon detecting a gaze thereof at the specific location, using an eye tracker.

12. The method according to claim 1, wherein said instructing the human subject to look at the specific location further comprises providing a visual and/or audio feedback selected to enhance a positive mental reaction at said human subject.

13. The method according to claim 1, wherein the specific location is a point or a direction in an environment in which the human subject is present.

14. The method according to claim 1, wherein the user interface comprises a display, and wherein the specific location is point on the display.

15. A system comprising:
a computer memory configured to receive human subject data associated with a mental or a physical condition related to a human subject;
a computer processor configured to automatically select a specific location based on said human subject data; and
a user interface configured to instruct the human subject, to look at a specific location, while thinking about something associated with said mental or physical condition.

16. The system according to claim 15, wherein the computer memory configured to receive said human subject data by instructing the human subject to discuss complaints regarding a physical condition or the mental condition thereof, while monitoring a change in line of sight of the human subject towards a representative location.

17. The system according to claim 15, wherein the specific location is calculated further based on the representative location.

18. The system according to claim 17, further comprising a knowledge base, wherein the specific location is calculated further based on the knowledge base which associates representative locations with respective mental conditions.

19. The system according to claim 15, wherein the user interface comprises a display, and wherein the specific location is point on the display.

20. A non-transitory computer readable storage medium comprising a set of instructions that, when executed, cause a computer processor to:
receive human subject data associated with a mental or a physical condition related to a human subject;
automatically select a specific location based on said human subject data; and
cause a user interface to instruct the human subject to look at a specific location, while thinking about something associated with said mental or physical condition.
